(19) United States
(12) Patent Application Publication
(10) Pub. No.: US 2021/0020283 A1
(43) Pub. Date: Jan. 21, 2021

(54) CLOUD-BASED METHOD AND SYSTEM FOR EXTRACTION AND DISPLAY OF CLINICALLY-RELEVANT ACTIONABLE PATIENT DATA FROM MULTIPLE ELECTRONIC MEDICAL RECORDS

(71) Applicants: Abinash Achrekar, Albuquerque, NM (US); Shalini Shanker, Albuquerque, NM (US)

(72) Inventors: Abinash Achrekar, Albuquerque, NM (US); Shalini Shanker, Albuquerque, NM (US)

(21) Appl. No.: 16/926,913

(22) Filed: Jul. 13, 2020

Related U.S. Application Data
(60) Provisional application No. 62/876,582, filed on Jul. 19, 2019.

(51) Int. Cl. G16H 15/00; G06F 16/26; G06F 16/25; G06F 16/23; G16H 10/60; G16H 50/70; G16H 10/40; G16H 50/20

(52) U.S. Cl. CPC G16H 15/00; G06F 16/26; G06F 16/252; G06F 16/2379; A61B 5/0022; G16H 50/70; G16H 10/40; G16H 50/20; G16H 10/60

(57) ABSTRACT

A method and system for managing and displaying medical data, can involve extracting clinically-relevant actionable patient data from at least one database among a plurality of databases containing varying medical data, displaying the clinically-relevant actionable patient data in a graphical user interface for a user, in response to a user input by the user, and automatically updating the clinically-relevant actionable patient data and overwriting the clinically-relevant actionable patient data with the updated clinically-relevant actionable patient data so as to reduce data storage and conserve memory space in a data-processing system.

### CROSS-REFERENCE TO PROVISIONAL APPLICATION

This patent application claims priority under 35 U.S.C. § 119(e) to U.S. Provisional Patent Application Ser. No. 62/876,582 entitled “Cloud-Based Method and System for Extraction and Display of Clinically-Relevant Actionable Patient Data from Multiple Electronic Medical Records,” which was filed on Jul. 19, 2020, the disclosure of which is incorporated herein by reference in its entirety.

### TECHNICAL FIELD

Embodiments are related to improvements in data-processing systems and methods. Embodiments further relate to the field of electronic medical records management, deployment, diagnosis, and display through a data-processing system.

### BACKGROUND

There are currently over 700 federally certified electronic health record (““EHR”) vendors. Electronic medical records were intended to bring higher-quality care and save costs by harnessing big data. This did not happen, however, in practice. Every hospital or clinic (provider) chooses an EHR for their respective practice, which may be isolated from other EHRs. When patients move between providers, practitioners cannot access patient data remotely. They must request patient health record from the provider. This can take an average of three days or more and the patient's information may arrive on hundreds of pages by fax or CD-ROM.

Clinicians typically cannot wait three days or more to take action in emergency settings, which often can result in repeat testing. Additionally, current file sharing systems are cumbersome and inundated with too much superfluous information.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for methods and systems for managing and displaying medical data.

It is another aspect of the disclosed embodiments to provide for a cloud-based, blockchain and Health Insurance Portability and Accountability Act (“HIPAA”) compliant method and system that uses machine learning and Artificial Intelligence (“AI”) to extract clinically-relevant actionable patient data from multiple electronic medical records for display through a simple graphical user interface.

It is further an aspect of the disclosed embodiments to provide for the storage of a patient's latest medical/laboratory test information, which automatically overwrites itself, thereby solving the issue of massive medical record storage.

The aforementioned aspects and other objectives and advantages can now be achieved as described herein. In an embodiment, a method for managing and displaying medical data, can involve: extracting clinically-relevant actionable patient data from at least one database among a plurality of databases containing varying medical data; displaying the clinically-relevant actionable patient data in a graphical user interface for a user, in response to a user input by the user; and automatically updating the clinically-relevant actionable patient data and overwriting the clinically-relevant actionable patient data with the updated clinically-relevant actionable patient data so as to reduce data storage and conserve memory space in a data-processing system.

In another embodiment, a system for managing and displaying medical data, can include at least one processor and a memory, the memory storing instructions to cause the at least one processor to perform: extracting clinically-relevant actionable patient data from at least one database among a plurality of databases containing varying medical data; displaying the clinically-relevant actionable patient data in a graphical user interface for a user, in response to a user input by the user; and automatically updating the clinically-relevant actionable patient data and overwriting the clinically-relevant actionable patient data with the updated clinically-relevant actionable patient data so as to reduce data storage and conserve memory space in a data-processing system.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a block diagram of a cloud-based system for extracting and displaying clinically-relevant actionable patient data from multiple electronic medical records, in accordance with an embodiment;

FIG. 2 illustrates a block diagram of the architecture of the MB module 103, in accordance with an embodiment;

FIG. 3 illustrates a chart depicting an example of patient problems and information needed, in accordance with an embodiment;

FIG. 4 illustrates a mobile computing device with a GUI (Graphical User Interface) screen that displays an example of patient data including emergency contact information along with physical, specialist, and other contact information, in accordance with an embodiment;

FIG. 5 illustrate a mobile computing device with a GUI screen that displays patient diagnosis, in accordance with an embodiment;

FIG. 6 illustrates a mobile computing device with patient diagnostic and information including radiology, cardiac and laboratory data, in accordance with an embodiment;

FIG. 7 illustrates a GUI window that displays patient diagnostic and other information, in accordance with an embodiment;

FIG. 8 illustrates a GUI window that displays patient information displayed as the result of a search, in accordance with an embodiment;

FIG. 9 illustrates a GUI window that can display information pertaining to a medical condition, in accordance with an embodiment;

FIG. 10 illustrates a schematic view of a computer system, in accordance with an embodiment; and

FIG. 11 illustrates a schematic view of a software system including a module, an operating system, and a user interface, in accordance with an embodiment.

### DETAILED DESCRIPTION

The particular values and configurations discussed in these non-limiting examples can be varied and are cited merely to illustrate one or more embodiments and are not intended to limit the scope thereof.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” or “in an embodiment” and variations thereof as utilized herein do not necessarily refer to the same embodiment and the phrase “in another embodiment” or “in another example embodiment” and variations thereof as utilized herein may or may not necessarily refer to a different embodiment. It is intended, for example, that claimed subject matter can include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Several aspects of data-processing systems will now be presented with reference to various systems and methods. These systems and methods will be described in the following detailed description and illustrated in the accompanying drawings by various blocks, modules, components, circuits, steps, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. Note that the term ‘data-processing system’ or ‘data-process apparatus’ as utilized herein can relate to a combination of machines, people, and processes that for a set of inputs produces a defined set of outputs. The inputs and outputs can be interpreted as data, facts, information etc. depending on the interpreter's relation to the system. Examples of a data-processing system or a data-processing apparatus can include a computing device, a server, a mobile computing device, or a combination of computing devices and components and modules (which may be hardware modules, software modules and/or combinations thereof).

By way of example, an element, or any portion of an element, or any combination of elements may be implemented with a “processing system”, a “data processing system” or a “data-processing apparatus” that can include one or more processors. Examples of processors can include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. One or more processors in the processing system or apparatus may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. A mobile “app” is an example of such software.

The term ‘processor’ as utilized herein can relate to an integrated electronic circuit that performs calculations that run a computer. A processor can perform arithmetical, logical, input/output (I/O) and other basic instructions that can be passed from an operating system (OS). Most other processes are dependent on the operations of a processor. The terms processor, CPU and microprocessor are commonly linked and may be utilized interchangeably with one another to refer to the same device, system or circuit.

Accordingly, in one or more exemplary embodiments, the functions described may be implemented in hardware, software, firmware, or any combination thereof. If implemented in software, the functions may be stored on or encoded as one or more instructions or code on a computer-readable medium. Computer-readable media includes computer storage media. Storage media may be any available media that can be accessed by a computer.

By way of example, and not limitation, such computer-readable media can include read-only memory (ROM) or random-access memory (RAM), electrically erasable programmable ROM (EEPROM), including ROM implemented using a compact disc (CD) or other optical disk storage, magnetic disk storage or other magnetic storage devices, or any other medium that can be used to carry or store desired program code in the form of instructions or data structures and that can be accessed by a computer. The term ‘disk’ and ‘disc’, as used herein, can include CD, laser disc, optical disc, digital versatile disc (DVD), and floppy disk where disks usually reproduce data magnetically, while discs reproduce data optically with lasers. Combinations of the above should also be included within the scope of computer-readable media. The term ‘computer-readable media’ can include storage devices such as flash drives or flash memory, and other types of memory devices.

The following discussion is intended to provide a brief, general description of suitable computing environments in which the system and method may be implemented. Although not required, the disclosed embodiments can be described in the general context of computer-executable instructions, such as program modules, being executed by a single computer. In most instances, a “module” (also referred to as an “engine”) may constitute a software application, but can also be implemented as both software and hardware (i.e., a combination of software and hardware).

Generally, program modules include, but are not limited to, routines, subroutines, software applications, programs, objects, components, data structures, etc., that perform particular tasks or implement particular data types and instructions. Moreover, those skilled in the art will appreciate that the disclosed method and system may be practiced with other computer system configurations, such as, for example, hand-held devices, multi-processor systems, data networks, microprocessor-based or programmable consumer electronics, networked PCs, minicomputers, mainframe computers, servers, and the like.

Note that the term module (or an engine) as utilized herein may refer to a collection of routines and data structures that perform a particular task or implements a particular data type. Modules may be composed of two parts: an interface, which lists the constants, data types, variable, and routines that can be accessed by other modules or routines, and an implementation, which is typically private (accessible only to that module) and which includes source code that actually implements the routines in the module. The term module may also simply refer to an application, such as a computer program designed to assist in the performance of a specific task, such as word processing, accounting, inventory management, etc.

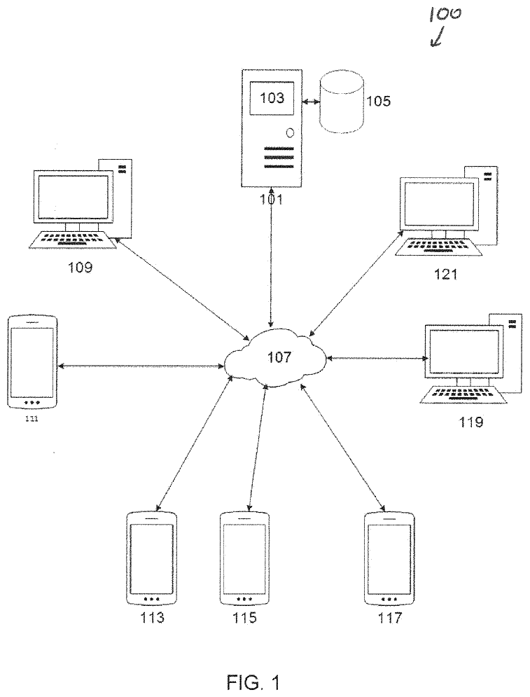

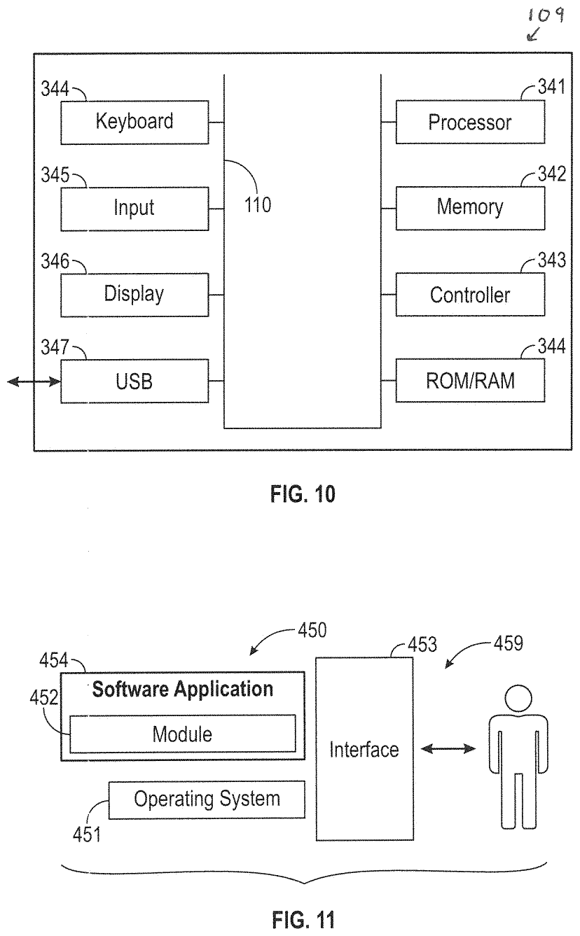

In some example embodiments, the term “module” can also refer to a modular hardware component or a component or element that can be a combination of hardware and software. It should be appreciated that implementation and processing of such modules according to the approach described herein can lead to improvements in processing speed and ultimately in energy savings and efficiencies in a data-processing system such as, for example, the data-processing system 109 shown in FIGS. 1 and 10. A “module” can thus perform the various steps, operations or instructions discussed herein to achieve the aforementioned improvements in processing speed and energy savings and efficiencies in a data-processing system.

FIG. 1 illustrates a block diagram of a cloud-based system 100 for extracting and displaying clinically relevant actionable patient data from multiple electronic medical records, in accordance with an embodiment. As will be discussed in greater detail herein, the system 100 can be implemented in the context of a cloud-based, blockchain, and HIPAA compliant module that uses machine learning and AI to extract clinically-relevant actionable patient data from multiple electronic medical records and display data with a simple user-interface. Such an approach can store the latest medical test and can automatically overwrite itself, thereby solving the issue of massive medical record storage.

Note that the term clinically-relevant actionable patient data as utilized herein can refer to data relating to the “bedside” of a patient, the course of his or her disease and/or the observation and treatment of patients directly. Such clinically-relevant actionable patient data can include patient data that a physician, nurse, physician's assistant, nurse practitioner or other medical personnel can utilize to chart and implement actionable treatment for the patient. The term clinically-relevant actionable patient data can also refer to data that is of clinical significance including the practical importance of a treatment effect—whether it has a real genuine, palpable, and/or noticeable effect on daily life.

Note that the term cloud-based as utilized herein relates to applications, services or resources made available to users on demand via the Internet from a cloud computing provider's servers. Cloud-based computing can be utilized as a way to increase capacity, enhance functionality or add additional services on demand without having to commit to potentially expensive infrastructure costs or increase/train existing in-house support staff. In addition, HIPAA is United States legislation that provides data privacy and security provisions for safeguarding medical information.

Referring now to FIG. 1, the system 100 can include a server 101 hosting a MB (Medicine Box) module 103 that can provide electronic access to a database 105. The database 105 can store patient medical records, including, for example, baseline records, incident records, prescriptions, treatment programs, and so on. The database 105 can further store a plurality of access permissions, which can define which types or classes of users may read or write data to the database 105. The database 105 may be distributed, so that the access permissions and the patient medical records may be stored in different databases. Alternatively, all storage for the system may be in a single database.

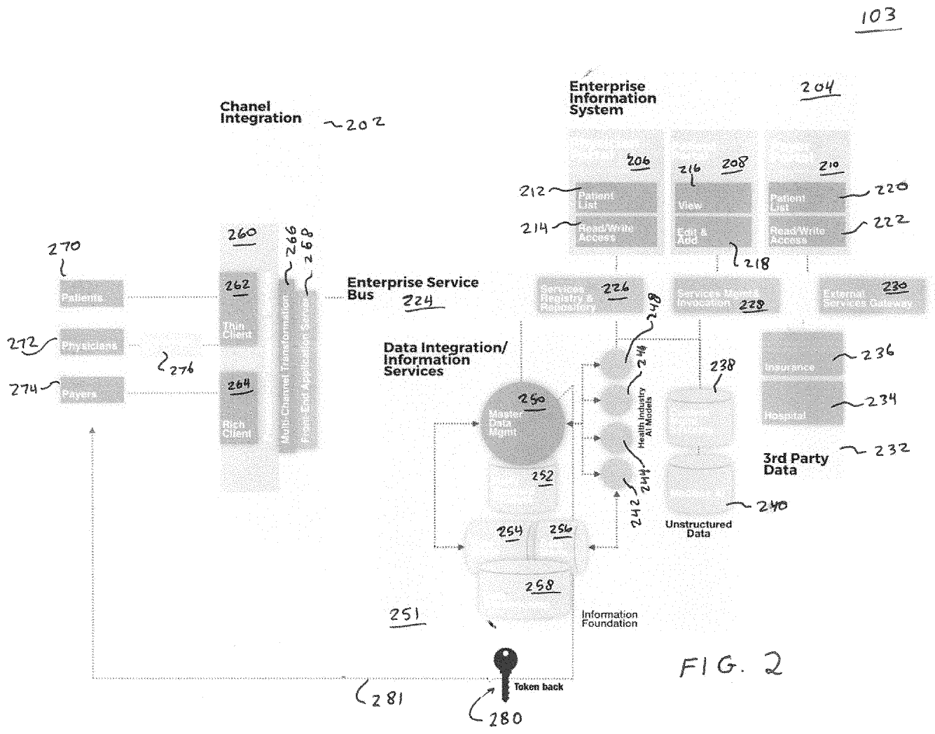

In a practical application, the database 105 may be configured from different databases and repositories. For example, the database 105 may include multiple databases, such as the databases 238, 240, 252, 254, 256, and 258 shown in FIG. 2. The server 101 can be linked by a network 107, such as the Internet, to one or more of the computing devices shown, such as, for example, computing devices 109, 121, 119 and mobile computing devices 111, 113, 115, and 117. Note that the term mobile computing device as utilized herein can refer to, for example, a smartphone, a tablet computing device, a wearable computing device (e.g., Apple Watch, Google Glass, and so on).

The MB module 103 can enable the various computing devices depicted in FIG. 1 to access the database 105, whether to contribute or retrieve patient data, depending on the permissions related to the access profile of the user utilizing the computing device. Although the MB module 103 may run as a single utility, in embodiments, it can present as different sub-utilities, each having distinct functionalities depending on the identity and access level of the user attempting to access the MB module 103. The MB module 103 may be hosted on the server 101, as shown, and accessed by the various users as a webpage, or the utility may be hosted on each or some of the devices shown in FIG. 1.

The computing devices 109, 119, 121 and 111, 113, 115, 117 are shown in FIG. 1 as being either a desktop computer or mobile telephone, or standalone server, but it will be appreciated that the devices may be embodied by any suitable computing device or combination of computing devices, such as, for example, desktop computer, laptop computer, mobile telephone, tablet or server computer. Each device comprises a processor and memory, the memory having stored thereon computer instructions, which when executed, can cause the device to execute tasks described herein. The MB module 103 may be accessed by a web portal generating web pages accessible by web browsers or web-enabled applications executed on devices such as computing devices 109, 119, 121 and 111, 113, 115, 117.

The network 107 may provide for wired or wireless communication between the server and the devices. Wireless communication may be provided by any suitable protocol, such as, for example, IEEE 802.11, GPRS, 3G, 4G, and LTE. The network 107 may be the Internet.

FIG. 2 illustrates a block diagram of the architecture of the MB module 103, in accordance with an embodiment. As shown in FIG. 2, the MB module 103 can provide for an enterprise information system 204 that offers a physician portal 206, a patient portal 208, and a payer portal 210. Note that the term portal as utilized herein can relate to a communication portal including a web-based portal accessible by computing devices such as the computing devices 109, 119, 121 and 111, 113, 115, and 117 shown in FIG. 1.

The physician portal 206 can include a module 212 and a module 214. The module 212 can provide a patient list for a physician and the module 214 can provide for read/write access for a physician. The patient portal 208 can include a module 216 that allows a patient to view data, and a module 218 that allows a patient to edit or add to such data. The payer portal 210 can include a module 220 that can provide a patient list, and a module 222 that provides for read/write access. Note that the term ‘portal’ as utilized herein can relate to a web portal, which can be a specially designed website that brings information from diverse sources, like emails, online forums and search engines, together in a uniform manner. Usually, each information source can have its own dedicated area on the page for displaying information; often, the user can configure which information sources are to be displayed. The term ‘portal’ as utilized herein can also refer simply to a portal or entryway to the enterprise information system 204.

The MB module 103 additionally can provide for an enterprise service bus 224 that can include a services registry and repository 226, services management and invocation 228, and an external services gateway 230. The services registry and repository 226 can be maintained within a database such as, for example, the database 105 shown in FIG. 1. The enterprise service bus 224 can communicate bidirectionally with the enterprise system 204 and also with a data integration/information services module 251. The enterprise service bus 224 can also communicate bidirectionally with a third party data module 232 that maintains and processes hospital data 234 and insurance data 236.

The data integration/information services module 251 can include a number of databases, modules and other features including a content management systems database 238 that communicates with an AI database 238. The data contained in the content management systems database 238 and the AI database 238 can include unstructured data. The data integration/information services module 251 can further include one or more AI modules 242, 244, 246, 248, which can implement health industry AI models.

The AI models 242, 244, 246, 248 can each be implemented as different types of machine learning modules. For example, AI module 242 may be implemented as a supervised and semi-supervised machine learning application, AI module 244 may be implemented as an unsupervised machine learning module, AI module 246 may be implemented as a reinforcement machine-learning module, and AI module 248 may be implemented as an anomaly detection module.

The term machine learning as utilized herein can relate to applications of AI that can provide a system with the ability to automatically learn and improve from experience without being explicitly programmed. A primary goal of machine learning is to allow a computer or data-processing system to learn automatically without human intervention or assistance and adjust actions accordingly. Machine learning enables analysis of massive quantities of data. While it generally delivers faster, more accurate results in order to identify profitable opportunities or dangerous risks, it may also require additional time and resources to train it properly. Combining machine learning with AI and cognitive technologies can make it even more effective in processing large volumes of information.

Any machine learning or training may be used to implement AI modules 242, 244, 246, and 248. A probabilistic boosting tree, support vector machine, neural network (e.g., deep learning), sparse auto-encoding classifier, Bayesian network, or other now known or later developed machine learning may be used. Any semi-supervised, supervised, or unsupervised learning may be used. Hierarchal or other approaches may be used. In one embodiment, the classification is by a machine-learnt classifier learnt with deep learning. As part of identifying features that distinguish between different outcomes, the classifier is also machine learnt. Any deep learning approach or architecture may be used. For example, a convolutional neural network may be used. The network may include convolutional, sub-sampling (e.g., max pooling), fully connected layers, and/or other types of layers. By using convolution, the number of possible features to be tested is limited. The fully connected layers operate to fully connect the features as limited by the convolution layer after maximum pooling. Other features may be added to the fully connected layers, such as non-imaging or clinical information. Any combination of layers may be provided. Hierarchical structures are employed, either for learning features or representation or for classification or regression. The computer-based decision support system employs a machine learning algorithm for automated decision making.

In alternative embodiments, a model other than a machine-learnt predictor can be used. Rule based (e.g., decision tree), reduced order (e.g., lumped parameter model of the coronary circulation system), or other models can be used to implement AI modules 242, 244, 246 and/or 248.

The data integration/information services module 251 can further include a master data management module 250 that can communicate with one or more databases such as a database 254, a database 246, a database 258, and a database 252 that maintains party, account, and product information.

The enterprise service bus 224 can further communicate with a channel integration module 202 that includes a module 260 that provides for presentation/interaction services, a module 266 that provides for multi-channel transformation, and a front-end application server 268 that can be implemented via a server such as, for example, the server 101 shown in FIG. 1. The module 260 can provide for presentation/interaction services based on the type of client (e.g., a rich client 264, a thin client 262, etc). A patient 270, a physician 272, and a payer 274 can access the MB module 103 via computing devices such as, for example, the computing devices 109, 119, 121 or 111, 113, 115, 117 shown in FIG. 1. In the case of the physician 272, the physician 272 may be required to provide a physician ID 276 to access the MB module 103.

In addition, a token 280 may be required for the patient 270, the physician 272, and/or the payer 274 to access the MB module 103. The token 280 can be implemented as a web application that uses tokens to authenticate the patient 270, the physician 272, and/or the payer 274 requesting access to the module 103. Arrow 281 and token 280 shown in FIG. 2 illustrates an example of a token application. One non-limiting example of a token application that can be utilized in the context of system 100 and MB module 103 is disclosed in U.S. Patent Application Publication No. 20190103969 entitled “Systems and Method for Preventing Excess User Authentication Token Utilization Conditions in an Enterprise Computer Environment,” which published on Apr. 4, 2019 to Yakov Faitelson, et al., and which is incorporated herein by reference in its entirety.

As discussed previously herein, there are currently over 700 federally certified EHR vendors. Electronic medical records were intended to bring higher-quality care and save money by harnessing big data. However, this did not happen in practice. Every hospital or clinic (providers) selects an EHR for their respective practice, which is isolated from other EHRs. When patients move between providers, practitioners cannot access patient data remotely. They must request patient health record from the provider. This can take an average of three days and the patient's information typically arrives on hundreds of pages by fax or CD-Rom.

In addition, clinicians cannot wait three days to take action in emergency settings, which may result in repeat testing. Additionally, the current file sharing systems are too cumbersome and inundated with too much superfluous information.

The MB module 103 thus moves away from big data to small specific relevant data. A team of doctors can create a list of key demographics and top problems that may occur in an emergency and compile and present the information they may need to take immediate action. The MB module 103 thus extracts only this information from numerous EHRs and displays the top problems and top indicators for each patient in one screen. The MB module 103 can store the latest test and can automatically overwrite itself solving the issue of massive medical record storage.

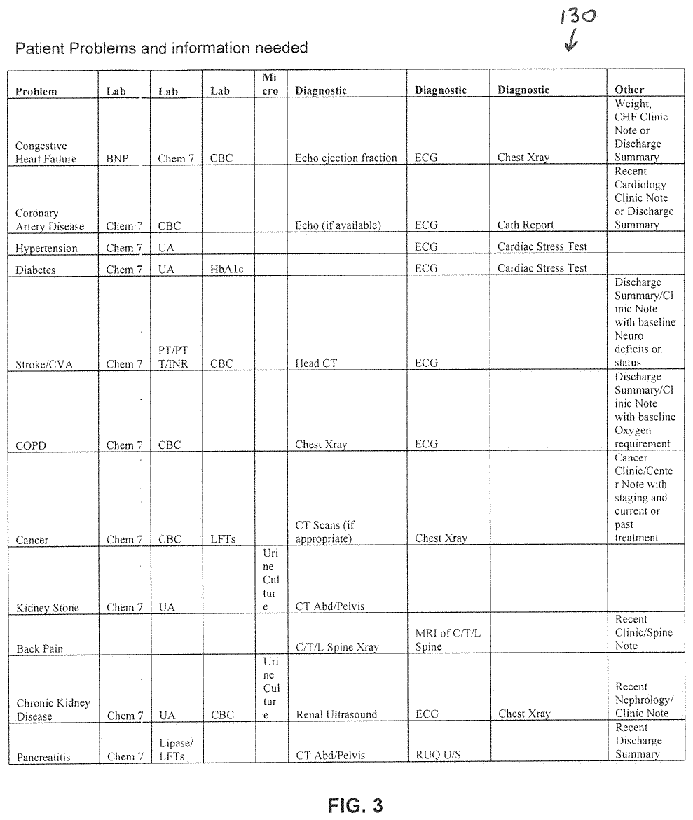

For example, if a patient has hypertension, the MB module 103 can extract the last Chem 7 labs, Urine Analysis, Electrocardiogram (ECG) and Cardiac Stress test data. This is information a doctor may require to know to assess and address the patient in a timely manner. FIG. 3 illustrates a chart 130 depicting an example of patient problems and information needed, in accordance with an embodiment.

The MB module 103 can offer a number of improvements and advantages.

For example, critical health information can be made more readily available by providing patients and doctors with access to key medical information on any patient, anytime and anywhere. Patients and/or approved family members can also share vital health information with providers via a mobile “app”.

The MB module 103 may also help to reduce so-called “doctor burnout” through the implementation of a better user experience and interface. The MB module 103 can synthesize information from numerous EHRs and display key and actionable information on a simple GUI instead of digging through massive patient databases. The information displayed helps practitioners direct care and make more specific medical searches if necessary from EHRs. In addition, the MB module 103 can facilitate increasing efficiency and reducing excessive repeat test by having the last or latest test data readily available. This also can reduce “upcoding” for Medicaid and Medicare by patients by having the latest patience test data readily available.

Another advantage of the MB module 103 can involve decreasing patient harm because patient medical records are now easily accessible to patients using an app on their phone or computer. Patients can check for inaccuracies and share information with EHRs.

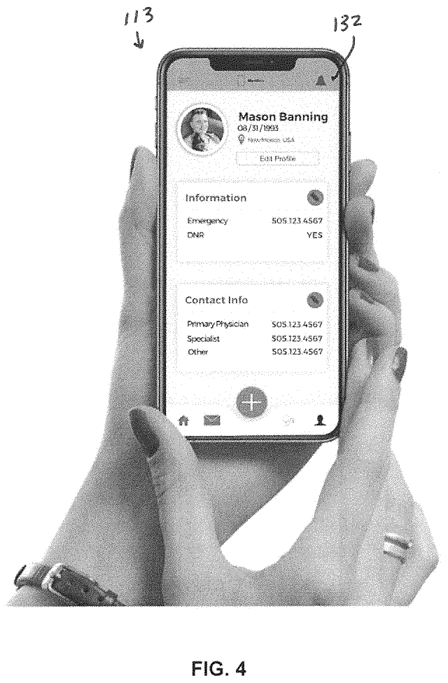

FIG. 4 illustrates a mobile computing device 113 with a GUI screen 132 that displays example patient data including emergency contact information and physical, specialist and other contact information, in accordance with an embodiment. Note that the term Graphical User Interface (“GUI”) refers to a form of a user interface that allows a user to interact with an electronic device such as the computing devices 109, 119, 121 or 111, 113, 115, 117 shown in FIG. 1 through graphical icons and visual indicators, menus, and so on, instead of text-based user interfaces, typed command labels or text navigation.

The GUI screen 132 shown in FIG. 4 can be provided by a GUI that can be configured for display and interactivity through a mobile computing device. The GUI screens shown in FIGS. 4-6 may be provided through a mobile application or “app” is downloadable to the mobile computing device 113. The mobile computing device 113, which may be a mobile phone, a tablet computing device, or other mobile device, can include a mobile operating system running on a processor (e.g., such as the processor 341 shown in FIG. 10). Mobile network connectivity can be provided via a wireless network communication interface, which can transmit and receive information via a wireless radiotelephone subsystem that can include an antenna, transceiver, and associated components to provide wireless communication connectivity via a mobile network to other mobile devices and to networked computers, such as computer servers, via the Internet and/or other networks.

A set of mobile applications (sometimes referred to individually as a “mobile app” or an “app”) can be stored on the mobile computing device 113 on a storage drive or other persistent storage device (not shown) and each is configured to run on top of mobile operating system, including by invoking services of the mobile operating system to communicate via a wireless network communication interface with remote resources, such as application servers running applications and/or services with which the mobile app is associated. The mobile operating system and mobile apps have access to and use a memory to store and retrieve data. For example, the mobile operating system may allocate to each app a region of memory to be used by that app to store app-related data. Similarly, each app may be allocated a set of logical locations in a persistent storage managed by the mobile operating system, e.g., an app-specific directory in a file system used by mobile operating system to manage persistently stored objects.

The mobile operating system of the mobile computing device 113 can be connected to and manages app interactions with a display subsystem to “display” that can include a touch-sensitive display device, for example, a capacitive or other display able to generate and provide to the mobile operation system, signals representative of single and/or multi-touch gestures, such as swiping (and the direction thereof), pinching in or out, dragging, and dropping. A mobile app may be configured to display app display pages, e.g., app user interface pages, content display pages, etc., via the display associated with the mobile computing device 113. A mobile app also may be configured to receive user input provided via a display, e.g., selection, dragging, dropping, and/or other user input associated with physical interactions with the touch-sensitive surface of the display.

Note that non-limiting examples of a mobile app and a mobile computing device are disclosed in U.S. Patent Application Publication No. 20190089826, entitled “Multilayer Mobile App Interface” which issued on Mar. 21, 2019 and is incorporated herein by reference in its entirety.

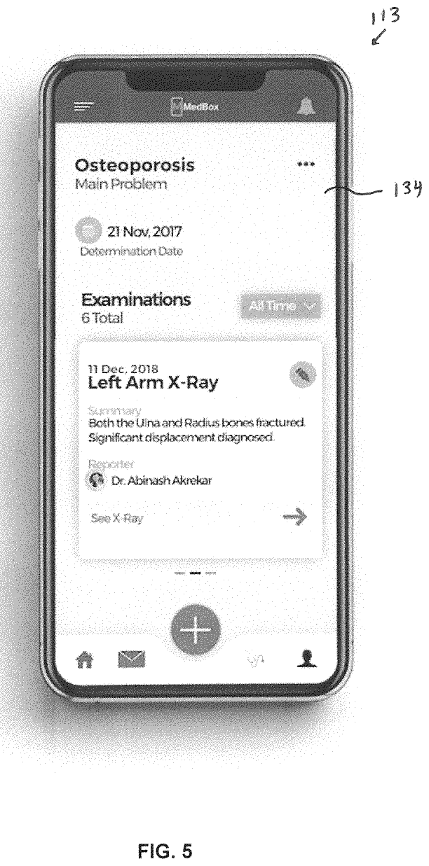

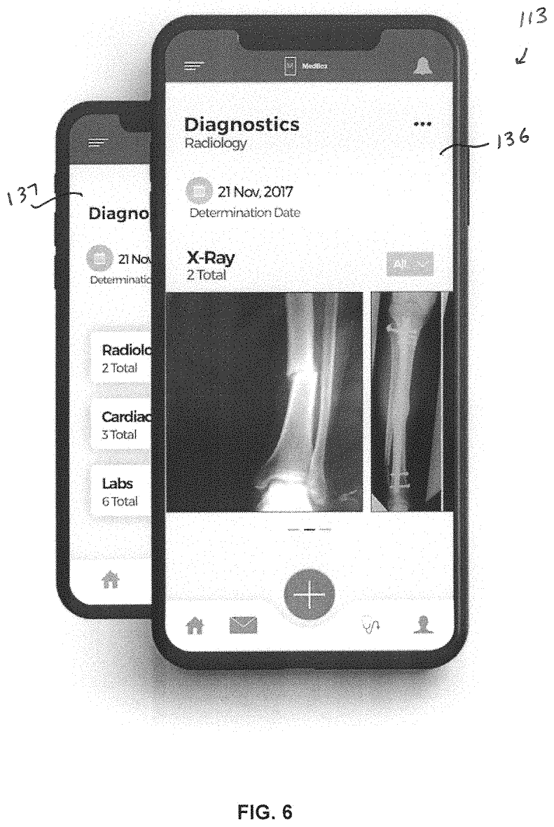

FIG. 5 illustrates the mobile computing device 113 with a GUI screen 134 that displays patient diagnosis information, in accordance with an embodiment. FIG. 6 illustrates the mobile computing device 113 with patient diagnostic information displayed in a GUI screen 136 and radiology, cardiac and laboratory data displayed in a GUI screen 137, in accordance with an embodiment.

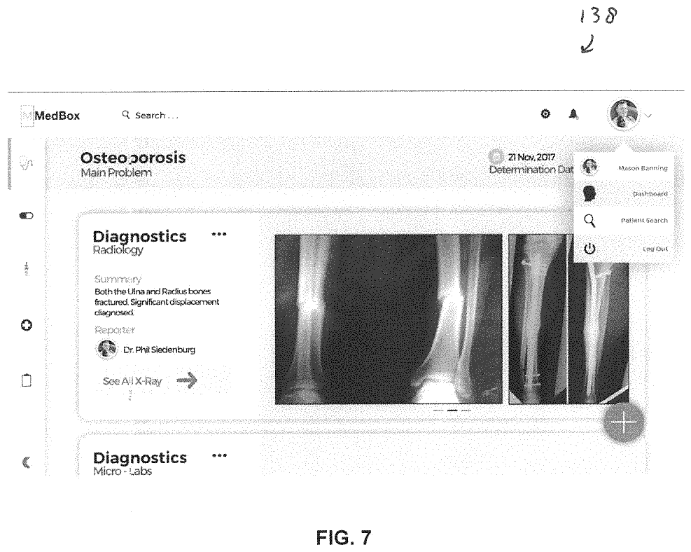

FIG. 7 illustrates a GUI window 138 that displays patient diagnostic and other information, in accordance with an embodiment. Note that the term window as utilized herein relates to a window of a windowing environment, which is an operating system or shell that presents a user with specially delineated areas of a GUI screen referred to as windows. Windowing environments typically allow windows to be resized and moved around on a display. The GUI windows shown in FIGS. 7-9 are examples of windows that can be provided by a windowing environment.

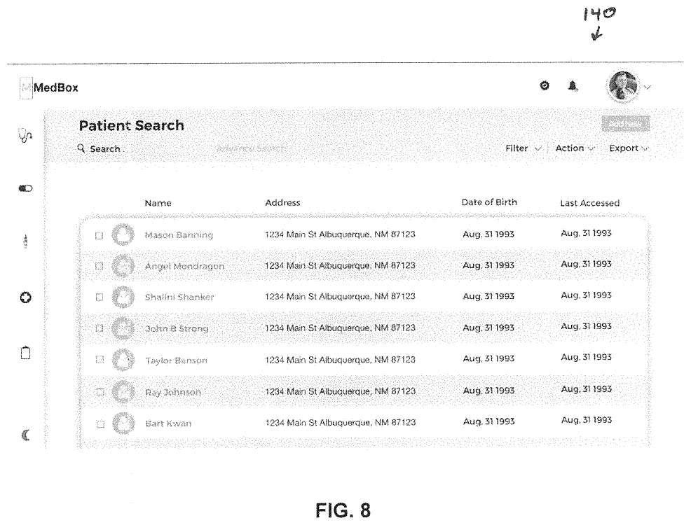

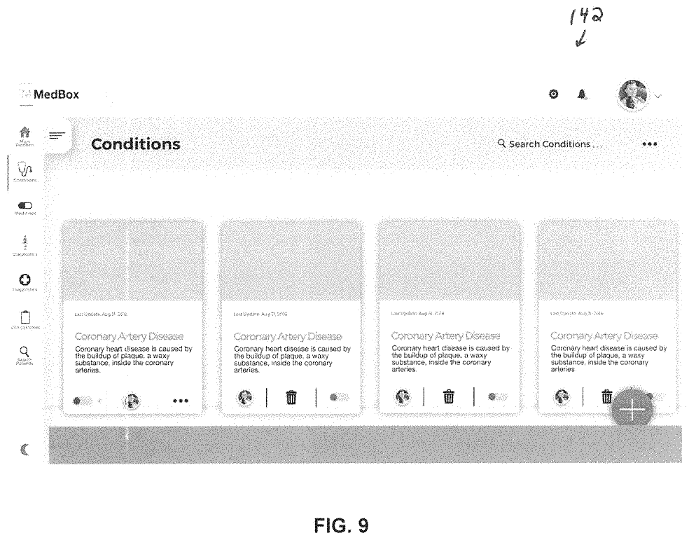

FIG. 8 illustrates a GUI window 140 that can display patient information displayed as the result of a patient search, in accordance with an embodiment. FIG. 9 illustrates a GUI window 142 that can display information pertaining to a medical condition, in accordance with an embodiment. In the example shown in FIG. 9, information pertaining to coronary artery disease is shown displayed.

The disclosed example embodiments are described at least in part herein with reference to flowchart illustrations and/or block diagrams of methods, systems, and computer program products and data structures according to embodiments of the invention. It will be understood that each block of the illustrations, and combinations of blocks, can be implemented by computer program instructions. These computer program instructions may be provided to a processor of, for example, a general-purpose computer, special-purpose computer, or other programmable data processing apparatus to produce a machine, such that the instructions, which execute via the processor of the computer or other programmable data processing apparatus, create means for implementing the functions/acts specified in the block or blocks.

To be clear, the disclosed embodiments can be implemented in the context of, for example a special-purpose computer or a general-purpose computer, or other programmable data processing apparatus or system. For example, in some example embodiments, a data processing apparatus or system can be implemented as a combination of a special-purpose computer and a general-purpose computer. In this regard, a system composed of different hardware and software modules and different types of GUI features may be considered a special-purpose computer designed with the specific purpose of rendering a visualization including the display of data through a GUI. In general, embodiments may be implemented as a method, and/or a computer program product at any possible technical detail level of integration. The computer program product may include a computer readable storage medium (or media) having computer readable program instructions thereon for causing a processor to carry out aspects of the embodiments.

The aforementioned computer program instructions may also be stored in a computer-readable memory that can direct a computer or other programmable data processing apparatus to function in a particular manner, such that the instructions (e.g., steps/operations) stored in the computer-readable memory produce an article of manufacture including instruction means which implement the function/act specified in the various block or blocks, flowcharts, and other architecture illustrated and described herein.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

The flow charts and block diagrams in the figures illustrate the architecture, functionality, and operation of possible implementations of systems, methods, and computer program products according to various embodiments (e.g., preferred or alternative embodiments). In this regard, each block in the flow chart or block diagrams may represent a module, segment, or portion of instructions, which comprises one or more executable instructions for implementing the specified logical function(s).

In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The functionalities described herein may be implemented entirely and non-abstractly as physical hardware, entirely as physical non-abstract software (including firmware, resident software, micro-code, etc.) or combining non-abstract software and hardware implementations that may all generally be referred to herein as a “circuit,” “module,” “engine”, “component,” “block”, “database”, “agent” or “system.” Furthermore, aspects of the present disclosure may take the form of a computer program product embodied in one or more non-ephemeral computer readable media having computer readable and/or executable program code embodied thereon.

FIGS. 10-11 are shown only as exemplary diagrams of data-processing environments in which example embodiments may be implemented. It should be appreciated that FIGS. 10-11 are only exemplary and are not intended to assert or imply any limitation with regard to the environments in which aspects or embodiments of the disclosed embodiments may be implemented. Many modifications to the depicted environments may be made without departing from the spirit and scope of the disclosed embodiments.

As illustrated in FIG. 10, some embodiments may be implemented in the context of a data-processing system 109, which can include, for example, one or more processors such as a processor 341 (e.g., a CPU (Central Processing Unit) and/or other microprocessors), a memory 342, a controller 343, additional memory such as ROM/RAM 332 (i.e. ROM and/or RAM), a peripheral USB (Universal Serial Bus) connection 347, a keyboard 344 and/or another input device 345 (e.g., a pointing device, such as a mouse, track ball, pen device, etc.), a display 346 (e.g., a monitor, touch screen display, etc) and/or other peripheral connections and components.

The system bus 110 serves as the main electronic information highway interconnecting the other illustrated components of the hardware of data-processing system 400. In some embodiments, the processor 341 may be a CPU that functions as the central processing unit of the data-processing system 400, performing calculations and logic operations required to execute a program. Read only memory (ROM) and random access memory (RAM) of the ROM/RAM 344 constitute examples of non-transitory computer-readable storage media.

The controller 343 can interface with one or more optional non-transitory computer-readable storage media to the system bus 110. These storage media may include, for example, an external or internal DVD drive, a CD ROM drive, a hard drive, flash memory, a USB drive or the like. These various drives and controllers can be optional devices. Program instructions, software or interactive modules for providing an interface and performing any querying or analysis associated with one or more data sets may be stored in, for example, ROM and/or RAM 344. Optionally, the program instructions may be stored on a tangible, non-transitory computer-readable medium such as a compact disk, a digital disk, flash memory, a memory card, a USB drive, an optical disc storage medium and/or other recording medium

As illustrated, the various components of data-processing system 109 can communicate electronically through a system bus 351 or similar architecture. The system bus 351 may be, for example, a subsystem that transfers data between, for example, computer components within data-processing system 109 or to and from other data-processing devices, components, computers, etc. The data-processing system 109 may be implemented in some embodiments as, for example, a server in a client-server based network (e.g., the Internet) or in the context of a client and a server (i.e., where aspects are practiced on the client and the server). The data-processing system 109 can be implemented as, for example, the computing device 109 shown in FIG. 1, or another type of computing device, such as, for example, the server 101 shown in FIG. 1.

In some example embodiments, data-processing system 191 may be, for example, a standalone desktop computer, a laptop computer, a Smartphone, a pad computing device and so on, wherein each such device is operably connected to and/or in communication with a client-server based network or other types of networks (e.g., cellular networks, Wi-Fi, etc).

FIG. 11 illustrates a computer software system 450 for directing the operation of the data-processing system 191 depicted in FIG. 10. The software application 454 may be stored, for example, in the memory 342 and/or another memory and can include one or more such as the module 452. The computer software system 450 also can include a kernel or operating system 451 and a shell or interface 453. One or more application programs, such as software application 454, may be “loaded” (i.e., transferred from, for example, mass storage or another memory location into the memory 342) for execution by the data-processing system 191. The data-processing system 191 can receive user commands and data through the interface 453; these inputs may then be acted upon by the data-processing system 191 in accordance with instructions from operating system 451 and/or software application 454. The interface 453 in some embodiments can serve to display results, whereupon a user 459 may supply additional inputs or terminate a session. The software application 454 can include module(s) 452, which can, for example, implement the steps, instructions or operations such as those discussed herein.

FIGS. 10-11 are intended as examples and not as architectural limitations of disclosed embodiments. Additionally, such embodiments are not limited to any particular application or computing or data processing environment. Instead, those skilled in the art will appreciate that the disclosed approach may be advantageously applied to a variety of systems and application software. Moreover, the disclosed embodiments can be embodied on a variety of different computing platforms, including Macintosh, UNIX, LINUX, and the like.

It is understood that the specific order or hierarchy of steps, operations, or instructions in the processes or methods disclosed is an illustration of exemplary approaches. For example, the various steps, operations or instructions discussed herein can be performed in a different order. Similarly, the various steps and operations of the disclosed example pseudo-code discussed herein can be varied and processed in a different order. Based upon design preferences, it is understood that the specific order or hierarchy of such steps, operation or instructions in the processes or methods discussed and illustrated herein may be rearranged. The accompanying claims, for example, present elements of the various steps, operations or instructions in a sample order, and are not meant to be limited to the specific order or hierarchy presented.

The inventors have realized a non-abstract technical solution to the technical problem to improve a computer-technology by improving efficiencies in such computer technology. The disclosed embodiments offer technical improvements to a computer-technology such as a data-processing system, and further provide for a non-abstract improvement to a computer technology via a technical solution to the technical problem(s) identified in the background section of this disclosure. The ability to provide patient information, for example, in a fast and efficient manner can lead to improved efficiencies in memory management and processing in the underlying computer technology. The embodiments disclosed herein can provide the benefits of a more seamless GUI implementation along with faster searching of databases and more efficient medical record data storage (i.e., reducing memory usage in a data-processing system). Such improvements can result from implementations of the disclosed embodiments. The claimed solution may be rooted in computer technology in order to overcome a problem specifically arising in the realm of computers, computer networks and data processing and data storage management.

It can be appreciated that a number of preferred and alternative embodiments are disclosed herein. For example, in an embodiment, a method for managing and displaying medical data, can involve extracting clinically-relevant actionable patient data from at least one database among a plurality of databases containing varying medical data; displaying the clinically-relevant actionable patient data in a graphical user interface for a user, in response to a user input by the user; and automatically updating the clinically-relevant actionable patient data and overwriting the clinically-relevant actionable patient data with the updated clinically-relevant actionable patient data so as to reduce data storage and conserve memory space in a data-processing system.

In an embodiment of the method, the clinically-relevant actionable patient data extracted from the at least one database containing the varying medical data can include patient medical test results.

In an embodiment of the method, the clinically-relevant actionable patient data extracted from the at least one database containing the varying medical data can comprise data created by a team of doctors indicative of key demographics and problems occurring in an emergency and information required to take immediate action with respect to a patient in the emergency.

In an embodiment of the method, the clinically-relevant actionable patient data extracted from the at least one database containing the varying medical data can comprise information from a group of electronic health records; and the clinically-relevant actionable patient data displayed in the graphical user interface can comprise top problems and top indicators for a single patient.

An embodiment of the method can further involve automatically overwriting the at least one database with latest test and medical information related to a patient.

In an embodiment of the method, the at least one database can comprise an AI (Artificial Intelligence) database that can include unstructured data and which can implement at least one health industry AI model.

In an embodiment of the method, the clinically-relevant actionable patient data can be displayed in the graphical user interface based on the type of patient.

In another embodiment, a system for managing and displaying medical data, can include at least one processor and a memory, the memory storing instructions to cause the at least one processor to perform: extracting clinically-relevant actionable patient data from at least one database among a plurality of databases containing varying medical data; displaying the clinically-relevant actionable patient data in a graphical user interface for a user, in response to a user input by the user; and automatically updating the clinically-relevant actionable patient data and overwriting the clinically-relevant actionable patient data with the updated clinically-relevant actionable patient data so as to reduce data storage and conserve memory space in a data-processing system.

In another embodiment, a system can include a plurality of databases containing varying medical data; and a graphical user interface that allows a user to access the plurality of databases, wherein clinically-relevant actionable patient data is extracted from at least one database among the plurality of databases containing the varying medical data, wherein the clinically-relevant actionable patient data is displayable in the graphical user interface for the user, in response to a user input by the user, and wherein the clinically-relevant actionable patient data is automatically updated and the clinically-relevant actionable patient data overwritten with the updated clinically-relevant actionable patient data to reduce data storage and conserve memory space in the system.

It will be appreciated that variations of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

What is claimed is:

1. A method for managing and displaying medical data, comprising:
extracting clinically-relevant actionable patient data from at least one database among a plurality of databases containing varying medical data;
displaying the clinically-relevant actionable patient data in a graphical user interface for a user, in response to a user input by the user; and
automatically updating the clinically-relevant actionable patient data and overwriting the clinically-relevant actionable patient data with the updated clinically-relevant actionable patient data so as to reduce data storage and conserve memory space in a data-processing system.

2. The method of claim 1 wherein the clinically-relevant actionable patient data extracted from the at least one database containing the varying medical data comprises patient medical test results.

3. The method of claim 1 wherein the clinically-relevant actionable patient data extracted from the at least one database containing the varying medical data comprises data created by a team of doctors indicative of key demographics and problems occurring in an emergency and information required to take immediate action with respect to a patient in the emergency.

4. The method of claim 1 wherein:
the clinically-relevant actionable patient data extracted from the at least one database containing the varying medical data comprises information from a group of electronic health records; and
the clinically-relevant actionable patient data displayed in the graphical user interface comprise top problems and top indicators for a single patient.

5. The method of claim 1 further comprising automatically overwriting the at least one database with latest test and medical information related to a patient.

6. The method of claim 1 wherein the at least one database comprises an AI (Artificial Intelligence) database that includes unstructured data and which implements at least one health industry AI model.

7. The method of claim 1 the clinically-relevant actionable patient data is displayed in the graphical user interface based on a type of patient.

8. A system for managing and displaying medical data, comprising:
at least one processor and a memory, the memory storing instructions to cause the at least one processor to perform:
extracting clinically-relevant actionable patient data from at least one database among a plurality of databases containing varying medical data;
displaying the clinically-relevant actionable patient data in a graphical user interface for a user, in response to a user input by the user; and
automatically updating the clinically-relevant actionable patient data and overwriting the clinically-relevant actionable patient data with the updated clinically-relevant actionable patient data so as to reduce data storage and conserve memory space in a data-processing system.

9. The system of claim 8 wherein the clinically-relevant actionable patient data extracted from the at least one database containing the varying medical data comprises patient medical test results.

10. The system of claim 8 wherein the clinically-relevant actionable patient data extracted from the at least one database containing the varying medical data comprises data created by a team of doctors indicative of key demographics and problems occurring in an emergency and information required to take immediate action with respect to a patient in the emergency.

11. The system of claim 8 wherein:
the clinically-relevant actionable patient data extracted from the at least one database containing the varying medical data comprises information from a group of electronic health records; and
the clinically-relevant actionable patient data displayed in the graphical user interface comprise top problems and top indicators for a single patient.

12. The system of claim 8 wherein the instructions are further configured to cause the at least one processor to perform automatically overwriting the at least one database with latest test and medical information related to a patient.

13. The system of claim 8 wherein the at least one database comprises an AI (Artificial Intelligence) database that includes unstructured data and which implements at least one health industry AI model.

14. The system of claim 8 the clinically-relevant actionable patient data is displayed in the graphical user interface based on a type of patient.

15. A system, comprising:
a plurality of databases containing varying medical data;
a graphical user interface that allows a user to access the plurality of databases;
wherein clinically-relevant actionable patient data is extracted from at least one database among the plurality of databases containing the varying medical data;
wherein the clinically-relevant actionable patient data is displayable in the graphical user interface for the user, in response to a user input by the user; and
wherein the clinically-relevant actionable patient data is automatically updated and the clinically-relevant actionable patient data overwritten with the updated clinically-relevant actionable patient data to reduce data storage and conserve memory space in the system.

16. The system of claim 15 wherein the clinically-relevant actionable patient data extracted from the at least one database containing the varying medical data comprises patient medical test results.

17. The system of claim 15 wherein the clinically-relevant actionable patient data extracted from the at least one database containing the varying medical data comprises data created by a team of doctors indicative of key demographics and problems occurring in an emergency and information required to take immediate action with respect to a patient in the emergency.

18. The system of claim 15 wherein:
the clinically-relevant actionable patient data extracted from the at least one database containing the varying medical data comprises information from a group of electronic health records; and
the clinically-relevant actionable patient data displayed in the graphical user interface comprise top problems and top indicators for a single patient.

19. The system of claim 15 wherein the at least one database is automatically overwritten with latest test and medical information related to a patient, and wherein the clinically-relevant actionable patient data is displayed in the graphical user interface based on a type of patient.

20. The system of claim 15 wherein the at least one database comprises an AI (Artificial Intelligence) database that includes unstructured data and which implements at least one health industry AI model.
